(19) United States
(12) Patent Application Publication
(10) Pub. No.: US 2005/0003360 A1
(43) Pub. Date: Jan. 6, 2005

(54) Array systems and methods

(76) Inventor: Ruo-Pang Huang, Norcross, GA (US)

Correspondence Address: Christopher B Linder;Thomas Kayden Horstemeyer & Risley, Suite 1750, 100 Galleria Parkway NW, Atlanta, GA, 30339-5948, US

(21) Appl. No.: 10/495,390

(22) PCT Filed: Nov. 13, 2002
(86) PCT No.: PCT/US02/36340

Related U.S. Application Data
(60) Provisional application No. 60/338,801, filed on Nov. 13, 2001.

(51) Int. Cl. C12Q 1/68

(52) U.S. Cl. 435/6

(57) ABSTRACT

Methods, kits, arrays, and biosensors for detecting proteins, modified-proteins, protein-protein interactions, protein-DNA interactions, autoantibodies, and protein-small molecule interactions, are disclosed. A representative method of detecting proteins of the present invention includes exposing a solid support to a solution containing proteins; conjugating proteins to the solid support; exposing the solide support to a plurality of types of DNA-conjugated antibodies, wherein each type of DNA-conjugated antibody has an affinity for a specified protein; forming a complex between a protein conjugated with the solid support and a type of DNA-conjugated antibody when the protein is the specified protein for which the DNA-conjugated antibody has an affinity; separating the complex from the solution of proteins and the DNA-conjugated antibodies; releasing DNA from the DNA-conjugated antibodies; and detecting the DNA, wherein each DNA indicates the presence of the specified proteins.

### CROSS-REFERENCE TO RELATED APPLICATION

This application claims priority to copending U.S. provisional application entitled, “Immuno-DNA Array Systems and Methods,” having Ser. No. 60/338,601, filed Nov. 13, 2001, which is entirely incorporated herein by reference.

### TECHNICAL FIELD

The present invention is generally related to the analysis of proteins and polypeptides and, more particularly, is related to systems and methods for the simultaneous detection of detecting proteins and protein interactions.

### BACKGROUND OF THE INVENTION

All cell functions, including cell proliferation, cell death, and all differentiation, as well as maintenance of health status and development of disease are controlled by many genes and signaling pathways. New techniques such as cDNA microarrays have enabled the analysis of the global gene expression. DNA microarray technology permits systematic approaches to biological discovery that has a profound impact on cancer research. The ability to obtain global gene expression profiles promises to be an exceptionally powerful means to explore basic biology, facilitate drug discovery, provide new diagnostic tools for diseases, and tailor therapeutics to specific gene profiles. For instance, in basic cancer biology, the global analysis of gene profiles in cancer cells can uncover crucial clues to the underlying changes in genetic networks and programs of malignantly transformed cells. In addition, DNA microarrays are useful for classifying human diseases. These types of gene profiles provide valuable information about the molecular mechanisms responsible for disease development, disease diagnosis, and patient prognosis.

Although DNA microarray analysis of global gene expressions holds great promise in the fight against human disease, proteins do almost all of the work in the cell.

Experimental evidence clearly shows a disparity between the relative expression levels of mRNA and their corresponding proteins. More importantly, protein-protein interactions, protein modification, and protein-DNA interactions are important concepts for studying how proteins perform their functions, which cannot be studied by DNA alone.

Protein analysis is thereof important to understanding these concepts. In some cases such as cytokines and growth factors, protein analysis is easier to perform than genomic analysis. Further, protein analysis may be the only effective way to analyze the specific antibody levels. Therefore, to effectively treat cancer, a complete picture of the protein profile is desired. Unfortunately, unlike the cDNA microarray technology, the methodology that allows detecting an entire pool of proteins does not exist.

Currently, two-dimensional polyacrylamide gel electrophoresis (two dimensional gel system) coupled with mass spectrometry is the mainstream approach to analyze multiple protein expressions. However, this approach suffers from several problems such as requiring sophisticated devices, having low sensitivity, and having a lack of a qualification process of the protein. Some proteins cannot be identified using this approach. For example, low molecular weight proteins are difficult to quantify. In addition, the detection limit of the two dimensional gel system is at the nanogram level.

Unfortunately, many important proteins express much lower levels than the two-dimensional gel system can detect. Therefore, a heretofore unaddressed need exists in the industry to develop a new approach to assess proteins.

### SUMMARY OF THE INVENTION

Embodiments of the present invention include methods, kits, arrays, and biosensors for detecting proteins, modified-proteins, protein-protein interactions, protein-DNA interactions, autoantibodies, and protein-small molecule interactions. A representative method of detecting proteins of the present invention includes exposing a solid support to a solution containing proteins; conjugating proteins to the solid support; exposing the solid support to a plurality of types of DNA-conjugated antibodies, wherein each type of DNA-conjugated antibody has an affinity for a specified protein; forming a complex between a protein conjugated with the solid support and a type of DNA-conjugated antibody when the protein is the specified protein for which the DNA-conjugated antibody has an affinity; separating the complex from the solution of proteins and the DNA-conjugated antibodies; releasing DNA from the DNA-conjugated antibodies; and detecting the DNA, wherein each DNA indicates the presence of the specified proteins.

A representative method of detecting protein-protein interactions includes exposing a solid support to a plurality first proteins, wherein the plurality of first proteins conjugate with the solid support; exposing the solid support to a second solution of proteins, wherein the proteins in the second solution are capable of conjugating with the plurality of first proteins; exposing the solid support to a plurality of types of DNA-conjugated antibodies, wherein each type of DNA-conjugated antibody has an affinity for a specified protein; forming a complex between a protein in the second solution that has conjugated with the first protein and a type of DNA-conjugated antibody when the protein is the specified protein for which the DNA-conjugated antibody has an affinity; separating the complex from the solution of first proteins, second proteins, and the DNA-conjugated antibodies; releasing DNA from the DNA-conjugated antibodies; and detecting the DNA, wherein each DNA indicates the presence of the specified proteins which indicates that the specified proteins interacted with the first protein.

A representative method of detecting protein-DNA interactions includes exposing a solid support to a first DNA having at least one portion, wherein the first DNA conjugates with the solid support; exposing the solid support to a solution of proteins, wherein the proteins in the solution are capable of conjugating with a portion of the first DNA; exposing the solid support to a plurality of types of DNA-conjugated antibodies, wherein each type of DNA-conjugated antibody has an affinity for a specified protein; forming a complex between a protein in the solution that has conjugated with a portion of the first DNA and a type of DNA-conjugated antibody when the protein is the specified protein for which the DNA-conjugated antibody has an affinity; separating the complex from the solution of proteins and the DNA-conjugated antibodies; releasing DNA from the DNA-conjugated antibodies; and detecting the DNA, wherein each DNA indicates the presence of the specified proteins, which indicates that the specified proteins interacted with the portion of DNA.

A representative method of detecting modified proteins includes exposing a solid support to a solution containing modified-proteins; conjugating modified-proteins to the solid support; exposing the solid support to a plurality of types of DNA-conjugated antibodies, wherein each type of DNA-conjugated antibody has an affinity for a specified modified-protein; forming a complex between a modified-protein conjugated to the solid support and a type of DNA-conjugated antibody when the modified-protein is the specified modified-protein for which the DNA-conjugated antibody has an affinity; separating the complex from the solution of modified-proteins and the DNA-conjugated antibodies; releasing DNA from the DNA-conjugated antibodies; and detecting the DNA, wherein each DNA indicates the presence of the specified modified-proteins.

A representative method of detecting autoantibodies includes exposing a solution containing proteins to a solution containing a plurality of autoantibodies, wherein each autoantibody has an affinity of a specified protein; forming a first complex between a protein in the solution and a type of autoantibody when the protein is the specified protein for which the autoantibody has an affinity; exposing the first complex to a solution containing a plurality of types of DNA-conjugated antibodies, wherein each type of DNA-conjugated antibody has an affinity for a specified protein; forming a second complex between first complex and a type of DNA-conjugated antibody when the protein is the specified protein for which the DNA-conjugated antibody has an affinity; exposing the second complex to a solution having a plurality of antibodies, wherein each type of antibody has an affinity for a specified autoantibody; forming a third complex between the second complex and a type of antibody when the autoantibody is the specified antibody for which the antibody has an affinity; separating the third complex from the solutions; releasing DNA from the DNA-conjugated antibodies; and detecting the DNA, wherein each DNA indicates the presence of specified autoantibodies.

A representative method of detecting protein-small molecule interaction includes exposing a support to a plurality small molecules, wherein the plurality of small molecules conjugate with the support; exposing the support to a second solution of proteins, wherein each of the proteins in the second solution are capable of conjugating with a specified small molecule; forming a first complex between the small molecule and a type of protein, when the small molecule is the specified small molecule for which the protein has an affinity; exposing the support to a plurality of types of DNA-conjugated antibodies, wherein each type of DNA-conjugated antibody has an affinity for a specified protein; forming a second complex between a protein conjugated to the support and a type of DNA-conjugated antibody, when the protein is the specified protein for which the antibody has an affinity; separating the second complex from the solution of proteins and the DNA-conjugated antibodies; removing DNA from the DNA-conjugated antibodies; and detecting the DNA, wherein each DNA indicates the presence of the specified proteins, which indicates that the specified proteins interacted with the small molecules.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIGS. 1A and 1B are schematic diagrams that illustrate a representative embodiment of an immuno-DNA assay system for detecting proteins.

FIGS. 2A and 2B are schematic diagrams that illustrate another representative embodiment of the immuno-DNA assay system for detecting protein modifications.

FIGS. 3A-3C are schematic diagrams that illustrate a representative embodiment of the immuno-DNA assay system for assessing protein-protein interactions.

FIGS. 4A-4C are schematic diagrams that illustrate a representative embodiment of the immuno-DNA assay system for assessing DNA-protein interactions.

FIGS. 5A and 5B are schematic diagrams that illustrate a representative embodiment of the immuno-DNA assay system for assessing proteins that inhibit protein-protein interactions.

FIGS. 6A-6C are schematic diagrams that illustrate a representative embodiment of the immuno-DNA assay system for assessing proteins that inhibit DNA-protein interactions.

FIGS. 7A-7C are schematic diagrams that illustrate a representative embodiment of the immuno-DNA assay system for assessing antibody-autoantibody interactions.

### DETAILED DESCRIPTION

Definition of Terms

“DNA” (deoxyribonucleic acid) generally refers to any polynucleotide. “DNA” includes, without limitation, single- and double-stranded DNA; DNA that is a mixture of single- and double-stranded regions; single- and double-stranded ribonucleic acid (RNA); RNA that is mixture of single- and double-stranded regions; and hybrid molecules comprising DNA and RNA that may be single-stranded or, more typically, double-stranded or a mixture of single- and double-stranded regions. In addition, “DNA” refers to triple-stranded regions comprising RNA or DNA, or both RNA and DNA. The term “DNA” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “DNA” embraces chemically, enzymatically, or metabolically modified forms of polynucleotides as typically found in nature, as well as the chemical forms of DNA and RNA characteristic of viruses and cells. “DNA” also embraces relatively short polynucleotides, often referred to as oligonucleotides.

“Protein” refers to any peptide, polypeptide, or protein comprising two or more amino acids joined to each other by peptide bonds or modified peptide bonds, (i.e., peptide isosteres). “Protein” refers to both short chains (commonly referred to as peptides, oligopeptides, or oligomers) and to longer chains generally referred to as proteins. “Protein” may contain amino acids other than the 20 gene-encoded amino acids. “Protein” includes amino acid sequences modified either by natural processes, such as post-translational processing, or by chemical modification techniques, which are well known in the art. Such modifications are described in basic texts and in more detailed monographs, as well as in a voluminous research literature.

The term “antibody” includes reference to antigen binding forms of antibodies (e.g., Fab, F(ab)₂). The term “antibody” frequently refers to a polypeptide substantially encoded by an immunoglobulin gene or immunoglobulin genes, or fragments thereof, which specifically bind and recognize an analyte (antigen). However, while various antibody fragments can be defined in terms of the digestion of an intact antibody, one of ordinary skill in the art will appreciate that such fragments may be synthesized de novo either chemically or by utilizing recombinant DNA methodology. Thus, the term antibody, as used herein, also includes antibody fragments such as single chain Fv, chimeric antibodies (i.e., comprising constant and variable regions from different species), humanized antibodies (i.e., comprising a complementarity determining region (CDR) from a non-human source) and heteroconjugate antibodies (e.g., bispecific antibodies). In particular, autoantibodies are antibodies that react with a constituent of the tissue of the animal.

The term “antigen” includes reference to a substance to which an antibody can be generated and/or to which the antibody is specifically immunoreactive. The specific immunoreactive sites within the antigen are known as epitopes or antigenic determinants. These epitopes can be a linear array of monomers in a polymeric composition-such as amino acids in a protein-or consist of or comprise a more complex secondary or tertiary structure. Those of ordinary skill in the art will recognize that all immunogens (i.e., substances capable of eliciting an immune response) are antigens; however some antigens, such as haptens, are not immunogens, but may be made immunogenic by being coupled to a carrier molecule. An antibody immunologically reactive with a particular antigen can be generated in vivo or by recombinant methods such as selection of libraries of recombinant antibodies in phage or similar vectors. See, e.g., Huse et al., Science 246: 1275-1281 (1989); and Ward, et al., Nature 341: 544-546 (1989); and Vaughan et al., Nature Biotech. 14: 309-314 (1996).

Discussion

The present invention provides immuno-DNA array systems and methods for the analysis of polypeptides and proteins (hereinafter “proteins”). Specifically, embodiments of the present invention include methods, kits, assays, and biosensors for detecting proteins, modified-proteins, protein-protein interactions, protein-DNA interactions, autoantibodies, and protein-small molecule interactions. For example, embodiments of the immuno-DNA array system are capable of determining the presence of a protein and/or protein modification and are also capable of assessing protein-protein interactions, DNA-protein interactions, inhibitor-protein interactions, inhibitor-DNA interactions, and autoantibodies.

Generally, the immuno-DNA array system involves immobilization of one or more specified proteins onto one or more solid supports. The specified proteins bound to the solid support are then contacted with one or more DNA-conjugated antibody types. Each of the DNA-conjugated antibody types conjugate (e.g., bond, bind, chemically attached or associated with) with certain specified proteins. In addition, the DNA conjugated to the DNA-conjugated antibody type are unique for each type of DNA-conjugated antibody and have a common sequences at both ends, which can be used for amplification of the DNA.

After separation of the protein-bound DNA-conjugated antibodies from unbound DNA-conjugated antibodies, the DNA is made to release from the antibody and separated from solution. Subsequently, each DNA type can be amplified from common primers and detected by hybridization to DNA array chips or membranes. Thus, by detecting a particular type of DNA, the immuno-DNA array system indirectly detects the presence of the corresponding specified proteins. In addition to assessing the specified proteins in solution, quantification of the amount of specified protein can be conducted using the immuno-DNA array system by correlating the amount of each DNA type to an amount of specified protein. Methods and techniques for immobilization and detection of agents such as proteins has been described in Chin et al. (U.S. Pat. No. 6,197,599) and Wohlstadter et al. (U.S. Pat. No. 6,140,045), both of which are incorporated herein by reference.

In other embodiments, the antibody included in the DNA-conjugated antibody can be substituted with peptides, proteins (e.g., which can bind to specific protein), DNA (e.g., Aptamers, which can bind to specific protein), ligands, receptors, mrRNAs, oligonucleotides, lectins, hormones, carbohydrates, lipids, small molecules, cells, drugs, and other capture reagents known in the art.

The immuno-DNA array system has several advantages over protein arrays. The immuno-DNA array system is more sensitive than protein arrays since pooymerase chain reaction (PCR) amplification steps are included in this system. The immuno-DNA array system is flexible and sensitive enough to detect from microgram to attogram levels of proteins, while protein arrays can only detect nanogram to picogram levels of a protein. Protein arrays (e.g., prepared by spotting protein onto glass slide or other solid support) are more difficult to prepare and store since the proteins are usually unstable. Immuno-DNA array systems/methods of the present invention, on the other hand, use DNA arrays (e.g., prepared by spotting DNA onto glass slides or other solid supports), which are much more stable than protein arrays. Furthermore, the immuno-DNA array systems/methods of the present invention allow detection of protein-protein interactions, protein-DNA interactions, and protein-small molecular interactions, in a manner that resembles in vivo configurations, while current protein arrays only detect those interactions in vitro. In addition, multiple rounds of experiments can be performed separately. Subsequently, the samples from the multiple rounds can be pooled and amplified using PCR. Therefore, in the methods of the present invention high-density arrays can be created.

As indicated above, embodiments of the immuno-DNA array system/methods are capable of determining the presence of one or more proteins (e.g., ten to thousands). In this regard, the immuno-DNA array system/methods can detect multiple proteins simultaneously at microgram to alltogram levels. Furthermore, the immuno-DNA array system/methods can detect protein modification, such as phosphorylation, glycosylation, oxidation, ubiquitination, and acetylation. Consequently, the immuno-DNA array system/method can facilitate the accurate profiling of disease phenotypes and accelerate the identification and characterization of protein expression patterns, which can be used to determine cellular pathways associated with disease development. In particular, assessing proteins using the immuno-DNA array systems/methods of the present invention can provide a broad understanding of disease development (e.g., infection diseases, cancer, and immunological diseases). Current diagnostic methods can only measure the change of one protein at one time, which greatly limits accurate diagnosis. Simultaneous detection of multiple antibodies, which can be correlated to specified proteins, can provide a better analysis and greatly reduce the cost of protein analysis.

Embodiment A

An embodiment of the immuno-DNA array method/system is capable of assessing the presence of one or more proteins in a solution. The immuno-DNA array system/method can be used in a kit or biosensor. In general, the immuno-DNA array system/method includes conjugating the proteins of interest to a solid support and then contacting the solid support to a DNA-conjugated antibody solution having multiple DNA-conjugated antibody types. The DNA bound to each DNA-conjugated antibody type is unique for each DNA-conjugate antibody type. In addition, each type of DNA-conjugate antibody has an affinity for a particular protein. Consequently, each DNA-conjugated antibody type binds to a corresponding protein on the surface of the solid support.

Thereafter, the protein may be removed from the solid support and the DNA released from the DNA-conjugated antibody. The DNA is separated from the solution, amplified, and detected. The detection of a type of DNA indicates the presence of a particular protein. In this manner, an assessment of the proteins present in the protein solution can be conducted.

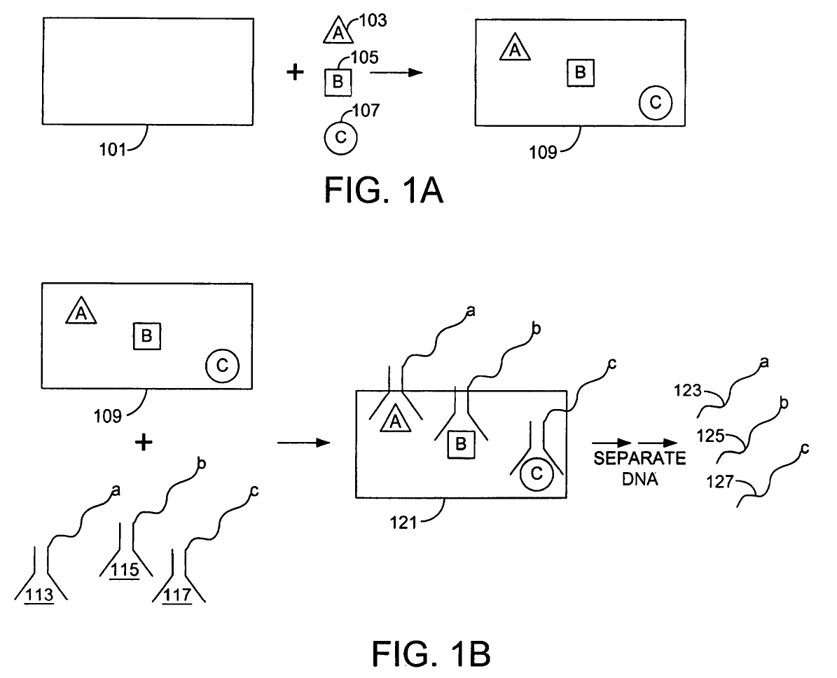

Now referring to the figures, FIGS. 1A and 1B are schematic diagrams that illustrate a representative embodiment of the immuno-DNA assay system/method. FIG. 1A is a schematic that illustrates contacting (e.g., incubating) a solution of proteins 103, 105, and 107 with a solid support 101. The solution of proteins can include one or more specified proteins 103, 105, and 107 (e.g., cytokine, EGF, insulin, MCP-1, EGFR, PDGFR, PLCγ or other solution containing proteins). In another embodiment, the solution of proteins can include one or more modified proteins (e.g., by phosphorylation, glycosylation, oxidation, ubiquitination, and acetylation) or other agents that can be used to identify protein modification. Further, the solution of proteins can include DNA, RNA, lectin, hormones, antibodies, carbohydrates, lipids, other organic chemicals, small molecules, cells, and/or drugs.

Some of the proteins in the solution 103, 105, and 107 conjugate with the solid support 101 forming a protein-conjugated solid support 109. The specified proteins 103, 105, and 107 can conjugate with the solid support 101 via covalent bonds and/or via non-covalent attractive forces such as hydrogen bond interactions, hydrophobic attractive forces, and ionic forces, for example.

The solid support 101 can be any solid support 101 that has an affinity for the proteins of interest 103, 105, and 107, and these include, for example, magnetic beads, agarose, membranes, sepharose, glass slides, and tissue culture plates. In addition, the solid support 101 may include compounds (e.g., proteins, carbohydrates, antibodies, etc.) bound to the surface of the solid support 101 that can enhance the affinity of the proteins of interest 103, 105, and 107 to conjugate to the solid support 101.

FIG. 1B is a schematic that illustrates contacting the protein-conjugated solid support 109 with a solution having a plurality of DNA-conjugated antibody types 113, 115, and 117. Each of the DNA-conjugated antibody types 113, 115, and 117 has an affinity for one or more specified proteins 103, 105, and 107, and preferably has an affinity for only one specified protein. In addition, the DNA bound to each DNA-conjugate antibody type 113, 115, and 117 is unique and corresponds with a specified protein 103, 105, and 107. Therefore, contacting the DNA-conjugated antibody solution with the protein-conjugated solid support 109 facilitates DNA-conjugated antibody types 113, 115, and 117 to bind or bond with a specified protein 103, 105, and 107 to form a DNA-protein-conjugated solid support 121.

The DNA-conjugated antibodies have the following features: 1) DNA and antibody have same specificity (e.g., epidermal growth factor (EGF) specific DNA conjugates to antibody against EGF or other specific sequence); 2) all DNA conjugated to antibodies contain common primers, therefore all DNA can be amplified with same pair of primers; 3) DNA can be conjugated to the antibody through covalent bond or noncovalent bond such as biotin-streptavidin interaction; and 4) DNA can be referred to as an expression sequence tag (EST), synthesized oligonuleotides, mRNA, or other genomic sequence.

The antibody can include, for example, a monoclonal antibody or a polyclonal antibody. In other embodiments the antibody can be substituted with peptides, proteins (e.g., which can bind to a specific protein), DNA (e.g., aptamers, which can bind to specific protein), ligands, receptors, mRNAs, oligonucleotides, lectins, hormones, carbohydrates, lipids, small molecules, cells, drugs, and other capture reagents known in the art.

Thereafter, the protein is removed from the DNA-protein-conjugated solid support 121 and the DNA 123, 125, and 127 is released from the DNA-conjugate antibody 113, 115, and 117. The DNA 123, 125, and 127 can be separated from solution and can be amplified and detected.

The removal of the protein from the DNA-protein-conjugated solid support 121 and release of the DNA 123, 125, and 127 from the DNA-conjugate antibody types can be executed using techniques known in the art, such as, for example, proteinase K digestion, endopeptidase digestion, aminopeptidase digestion, carboxypeptidase digestion, phenol extraction, chloroform extraction, and heat.

Each DNA type 123, 125, and 127 can be amplified using techniques known in the art to amplify DNA such as, for example PCR. In this regard, the DNA is amplified by the use of short synthetic oligonucleotides that are complementary to two terminal regions of the DNA. These oligonucleotides are extended by a thermostable DNA polymerase on the DNA template. This causes new DNA chains to span the region delineated by the two chosen termini. Consequently, a 100,000-fold or better amplification of the DNA can be achieved. In particular, each DNA type can be amplified and labeled by PCR in the presence of a pair of primers and labeled (e.g., cy3, cy5, 32p, 33p) using nucleotides, or other labeled nucleotides known in the art (e.g., biotin-labeled nucleotide). Since all DNA conjugated to antibodies contain identical sequence at both sides, the same primers can be used to amplify all DNA fragments simultaneously.

After amplification, each amplified DNA product is hybridized to a DNA chip or DNA array. The DNA chip can be prepared from oligonucleotides or DNA clones. Similarly, the DNA array can be prepared from oligonucleotides or DNA clones. Generally, since the identities of DNA in each spot are known, via hybridization of the DNA, the identities of DNA from DNA-conjugated antibodies can be deduced. The hybridization signals can be detected using techniques known in the art for detecting DNA such as, for example, fluorescence, chemiluminescence, substrate staining, and isotope detection. In addition, the identity of DNA released from DNA-conjugated antibodies can also be identified by other methods in the art such as, but not limited to, DNA sequence, electrophoresis, and techniques that use specific tags. The signals can also be amplified by other well-known methods, such as, but not limited to, rolling circle amplification (RCA), methods using biotinyl tyramide and hydrogen peroxide, and 3 DNA-label technology.

Each type of DNA 123, 125, and 127 corresponds to a specified protein 103, 105, and 107. In this manner, each type of DNA can be related to a specified protein indicating that the specified proteins 103, 105, and 107 are present in the protein solution.

In addition, the amount of each DNA 123, 125, and 127 can be quantified, which in turn can be related to the quantity of each of the specified proteins 103, 105, and 107 present in the solution of proteins.

Similarly, cells also can be conjugated to a solid support. After contacting with DNA-conjugated antibodies, the specific proteins present in the cell surface can be identified as described above.

Furthermore, specific protein modification can also be detected by this embodiment. In this regard, antibodies against specific modification, such as tyrosine phosphorylation, can be conjugated to a solid support. Tyrosine-phosphorylated proteins, for example, are separated from the unphosphorylated proteins after binding to the solid support conjugated with antibody against tyrosine-phosphorylation. The specific modified proteins are detected by contacting a solution containing a plurality of DNA-conjugated antibody types described above.

Embodiment B

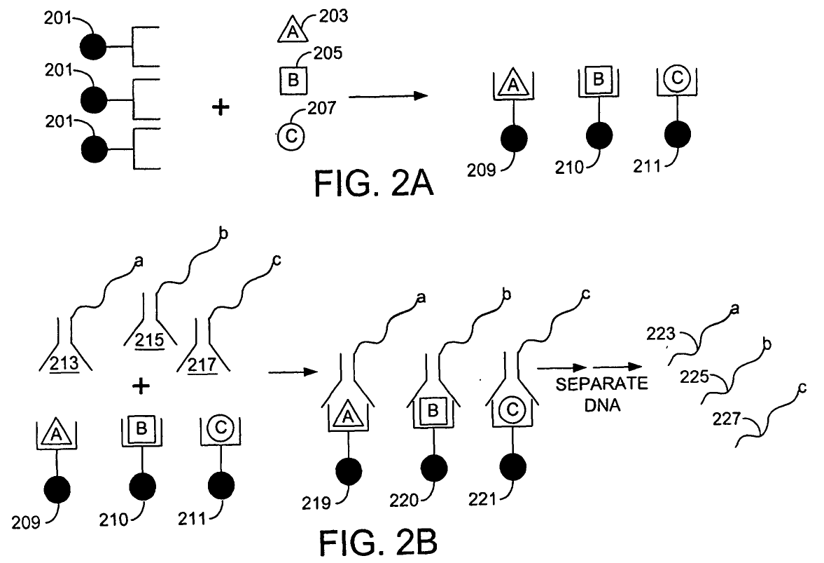

FIGS. 2A and 2B are schematic diagrams that illustrate another representative embodiment of the immuno-DNA assay method/system. The immuno-DNA array system can be used in a kit or biosensor. This embodiment includes multiple solid substrates 201 for the specified proteins 203, 205, and 207 to conjugate with, rather than one solid substrate as shown in FIGS. 1A-1B. FIG. 2A is a schematic that illustrates contacting multiple solid supports 201 with a solution of proteins that includes specified proteins 203, 205, and 207. Specified proteins 203, 205, and 207 conjugate with the solid supports 201 forming multiple protein-conjugated solid supports 209, 210, and 211. The solid support 201 can be any solid support that has an affinity for the specified proteins of interest and these include, for example, magnetic beads, agarose, membranes, and sepharose. The solution of proteins is similar to the solution described in relation to FIGS. 1A and 1B.

FIG. 2B is a schematic that illustrates contacting the protein-conjugated solid supports 209, 210, and 211 with a solution having a plurality of DNA-conjugated antibody types 213, 215 and 217. Each type of the DNA-conjugated antibody 213, 215, and 217 has an affinity for one or more specified proteins 203, 205 and 207, and preferably for only one specified protein. Therefore, contacting the DNA-conjugated antibody solution with the protein-conjugated solid supports 209, 210, and 211 facilitates the binding or bonding of each type of DNA-conjugated antibody 213, 215, and 217 with the specified proteins to form plural complexes containing DNA-conjugated antibody and specific protein-bound solid supports 219, 220, and 221. Unbound DNA-conjugated antibodies and the excess amount of DNA-conjugated antibodies are separated from the DNA-conjugated antibodies and specific protein complexes. Subsequently, the protein is removed from the DNA-protein-conjugated solid supports 219, 220, and 221 and the DNA 223, 225, and 227 is released from the DNA-conjugated antibody. The DNA 223, 225, and 227 is separated from the solution and can be amplified and detected in a manner consistent with the techniques discussed in reference to FIGS. 1A and 1B.

Embodiment C

In addition to identifying and quantifying specified proteins in a solution, the embodiments of the immuno-DNA array method/system can assess protein-protein interaction. The immuno-DNA array system can be used in a kit or biosensor. In this embodiment the immuno-DNA array system/method includes conjugating a first protein to a solid support forming protein-conjugated solid supports. Then the protein-conjugated solid support, is contacted with a protein solution having specified proteins. The specified proteins bind to the first protein forming protein-protein-conjugated solid supports. The protein-protein-conjugated solid supports are contacted with a DNA-conjugated antibody solution having multiple DNA-conjugated antibody types. The DNA bound to each DNA-conjugated antibody type is unique to that DNA-conjugate antibody type. Each type of DNA-conjugated antibody has an affinity for a particular specified protein. Consequently, each DNA-conjugated antibody type binds to a corresponding specified protein, which forms a complex on the surface of the protein-protein-conjugated solid support (DNA-protein-protein-conjugated solid supports). Thereafter, the protein is removed from the DNA-protein-protein-conjugated solid support and the DNA is released from the DNA-conjugated antibody. The DNA is separated from the solution and amplified and detected. Each type of DNA detected corresponds to a particular protein, so that detection of a type of DNA indicates that the specified protein interacted with the first protein. In this manner, an assessment of protein-protein interaction can be conducted.

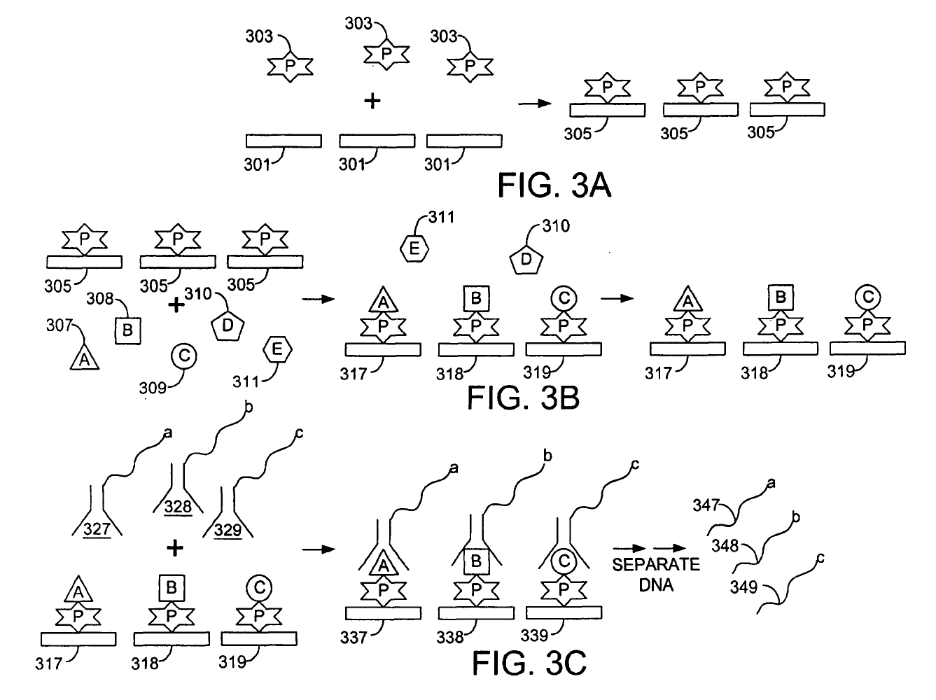

FIGS. 3A-3C are schematic diagrams that illustrate a representative embodiment where the immuno-DNA assay system/method can be used to assess protein-protein interactions. FIG. 3A is a schematic that illustrates contacting multiple solid supports 301 with a first protein 303 forming protein-conjugated solid supports 305.

FIG. 3B is a schematic that illustrates contacting the protein-conjugated solid supports 305 with a solution of proteins. The solution of proteins includes one or more specified proteins 307, 308, 309, 310, and 311. In another embodiment, the solution of proteins can include one or more small molecules. Contacting the protein-conjugated solid supports 301 with the solution of proteins facilitates the assessment of the interaction between the first protein 303 and the specified proteins 307, 308, 309, 310, and 311 present in solution. Subsequently, multiple protein-protein-conjugated solid supports 317, 318, and 319 are created with non-conjugated proteins 311 and 310 remaining in solution, which can be washed away.

FIG. 3C is a schematic that illustrates contacting the protein-protein solid supports 317, 318, and 319 with multiple DNA-conjugated antibody types 327, 328, and 329. Each of the DNA-conjugated antibody types 327, 328, and 329 has an affinity for one or more specified proteins 307, 308, 309, 310, and 311, and preferably only one specified protein. Contacting the DNA-conjugated antibody solution with the protein-protein solid supports 317, 318, and 319 facilitates DNA-conjugated antibody types 327, 328, and 329 to bond or bind with a specified protein to form DNA-protein-protein conjugated solid supports 337, 338, and 339. Thereafter, the first protein is removed from the DNA-protein-protein-conjugated solid supports 337, 338, and 339 and the DNA 347, 348, and 349 are released from the DNA-conjugated antibody. The DNA 347, 348, and 349 are separated from solution and can be amplified and detected in a manner consistent with the techniques discussed in reference to FIGS. 1A and 1B.

The same approach also can be used to detect and screen drug targets (small molecules) and protein interaction. In this embodiment, the drug can be conjugated to a solid support directly or indirectly through another molecule, such as albumin. The drug conjugated to the solid support then can be contacted with a solution having proteins or other molecules that may bind to the drug, such as lysate containing multiple proteins.

The detection of specific proteins interacting with the drug can be performed by the techniques discussed above.

Embodiment D

Another embodiment of the immuno-DNA array method/system is capable of assessing DNA-protein interactions. The immuno-DNA array system/method can be used in a kit or biosensor. In this embodiment the immuno-DNA array system/method includes conjugating a first DNA (e.g., usually a promoter from a particular gene) to a solid support forming a DNA-conjugated solid support. Then the DNA-conjugated solid support is contacted with a protein solution having specified proteins. The specified proteins conjugate with the first DNA, forming protein-DNA-conjugated solid supports. The protein-DNA-conjugated solid supports are contacted with a DNA-conjugated antibody solution having multiple DNA-conjugated antibody types. The DNA (i.e., the second DNA) bound to each DNA-conjugated antibody type is unique to that DNA-conjugate antibody type. Each type of DNA-conjugated antibody types has an affinity for a particular specified protein. Consequently, each DNA conjugates with a corresponding specified protein on the surface of the protein-DNA-conjugated solid support to form DNA-protein-DNA-conjugated solid supports. Thereafter, the complexes are separated from the unbound molecules and the excess DNA-conjugated antibodies. Second DNA are released from the DNA-conjugated antibodies and separated from the solution and amplified and detected. Since the first DNA does not contain common primers, it cannot be amplified under the same condition. Each type of second DNA detected corresponds to a specified protein, so that detection of a type of second DNA indicates that the specified protein interacted with the first DNA. In this manner, an assessment of the DNA-protein interaction can be conducted.

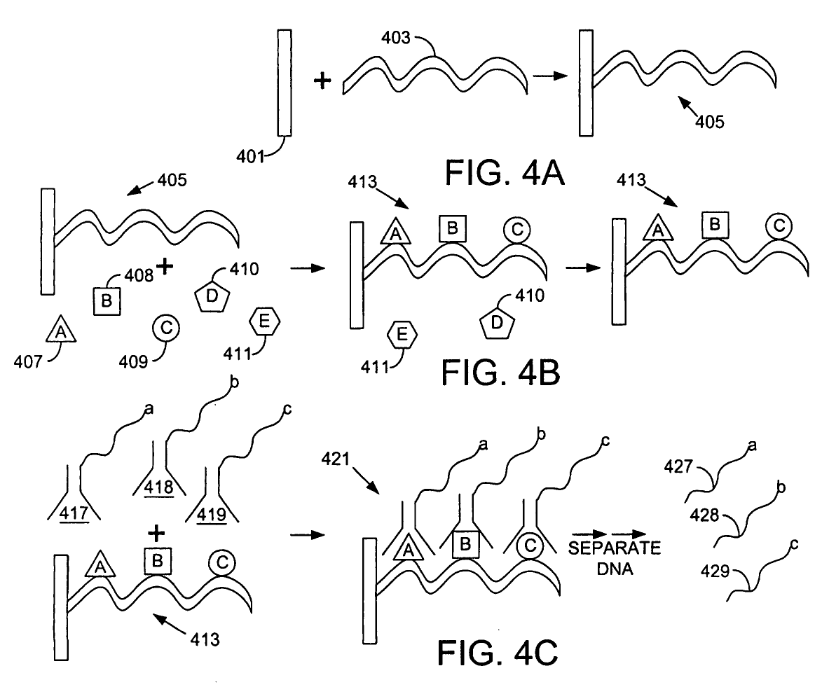

FIGS. 4A-4C are schematic diagrams that illustrate a representative embodiment of the immuno-DNA assay system that can be used to assess DNA-protein interactions. FIG. 4A is a schematic that illustrates the conjugation of a solid support 401 with the first DNA or DNA fragments 403 forming DNA-conjugated solid supports 405. In another embodiment, the first DNA 403 can be a polynucleotide as described above.

FIG. 4B is a schematic that illustrates contacting DNA-conjugated solid supports 405 with a solution of proteins. The solution of proteins includes multiple specified proteins 407, 408, 409, 410, and 411. Contacting the DNA-conjugated solid supports 405 with the solution of proteins facilitates the assessment of the interaction between the first DNA 403 and the multiple specified proteins 407, 408, 409, 410, and 411 in solution. Thereafter, multiple protein-DNA-conjugated solid supports 413 are created, where specified proteins 407, 408, and 409 can bind to particular areas of the DNA 403.

FIG. 4C is a schematic that illustrates contacting the protein-DNA solid supports 413 with multiple DNA-conjugated antibody types 417, 418, and 419. Each type of the DNA-conjugated antibody 417, 418, and 419 has an affinity for one or more specified proteins 407, 408, 409, 410, and 411, preferably only one specified protein. Contacting the DNA-conjugated antibody solution with the protein-DNA-conjugated solid supports 413 facilitates the bonding or binding of the DNA-conjugated antibody types 417, 418, and 419 with a specified protein 407, 408, 409, 410, and 411 to form a DNA-protein-DNA-conjugated solid support 421. Thereafter, the complexes are separated from the unbound proteins and excess DNA-conjugated antibody by precipitation, extensive wash, or magnetic force. DNA 427, 428, and 429 associated with the DNA-conjugated antibody are released and separated from solution and can be amplified and detected in a manner consistent with the techniques discussed in reference to FIG. 1A and 1B.

Embodiment E

Another embodiment of the immuno-DNA array method/system is capable of assessing one or more protein inhibitors. The immuno-DNA array system/method can be used in a kit or biosensor. In this regard, the immuno-DNA array system/method is capable of assessing if one or more inhibitors can inhibit known protein-protein interactions. In this embodiment the immuno-DNA array system/method includes conjugating a first protein to a solid support, forming protein-conjugated solid supports. The protein-conjugated solid supports are then contacted with a protein solution having specified proteins and inhibitor(s). The first protein and the specified proteins are known to interact under known incubation conditions. By adding the inhibitor, the immuno-DNA array system/method can assess whether the inhibitor(s) inhibit the interaction of one or more of the specified proteins with the first protein.

The specified protein and inhibitor are allowed to conjugate with the first protein-forming protein-protein-conjugated and inhibitor-protein-conjugated solid supports. The solid supports are contacted with a DNA-conjugated antibody solution having multiple DNA-conjugated antibody types. The DNA bound to each DNA-conjugated antibody type is unique to that DNA-conjugate antibody type. Each type of DNA-conjugated antibody has an affinity for a particular specified protein. Consequently, each DNA-conjugated antibody type binds with a corresponding protein on the surface of the protein-protein-conjugated solid support, forming DNA-protein-protein-conjugated solid supports.

Thereafter, the complexes are separated from first protein and the DNA is released from the DNA-conjugated antibody. The DNA is separated from the solution and amplified and detected. Each type of DNA detected corresponds to a particular protein, so that detection of a type of DNA indicates that the inhibitor proteins did not inhibit the protein-protein interaction. In this manner, an assessment of whether inhibitors inhibit particular protein-protein interactions can be conducted.

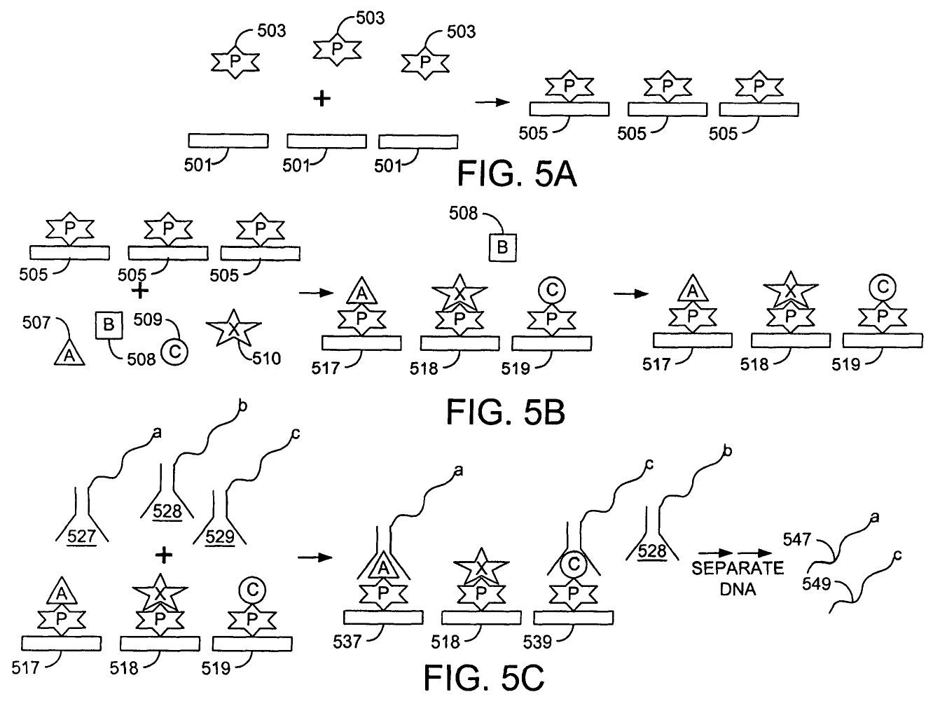

FIGS. 5A and 5B are schematic diagrams that illustrate a representative embodiment of the immuno-DNA assay system/method that can be used to assess the ability of an inhibitor to inhibit protein-protein interactions. After a protein-protein interaction is known for a pair of proteins (e.g., a first protein and a second protein), an assessment can be made to determine inhibitors that inhibit the interaction between the pair of ther first and second proteins.

FIG. 5A is a schematic that illustrates contacting multiple solid supports 501 with a solution of first proteins 503 forming protein-conjugated solid supports 505.

FIG. 5B is a schematic that illustrates contacting the first protein-conjugated solid supports 505 with a solution of proteins. The solution of proteins includes multiple target proteins 507, 508, and 509 and an inhibitor 510. The target proteins 507, 508, and 509 and the first protein 503 exhibit interaction under known incubation conditions. The inhibitor usually is a small molecule that is being assessed to determine whether or not it inhibits the interaction between the target proteins 507, 508, and 509 and the first protein 503. In this regard, contacting the first protein-conjugated solid supports 505 with the solution of target proteins 507, 508, and 509 and inhibitor 510 facilitates the formation of protein-protein-conjugated solid supports 517 and 519 and inhibitor-protein-conjugated solid supports 518. The remaining solution can be washed away at this time.

FIG. 5C is a schematic that illustrates contacting the protein-protein-conjugated solid supports 517 and 519 and inhibitor-protein-conjugated solid supports 518 with one or more DNA-conjugated antibody types 527, 528, and 529. Each of the DNA-conjugated antibody types 527, 528, and 529 has an affinity for one or more target proteins 507, 508, and 509, and preferably for only one target protein. Therefore, contacting the DNA-conjugated antibody types 527, 528, and 529 with the protein-protein-conjugated solid supports 517, 518, and 519 facilitates bonding or binding of the DNA-conjugated antibodies 527, 528, and 529 with a target protein 507, 508, and 509 to form DNA-protein-protein-conjugated solid supports 537 and 539. The inhibitor 510 has inhibited the target protein 508 from interacting with the first protein 503. Consequently, DNA-conjugated antibody 528 cannot conjugate to protein-protein-conjugated solid support 518. Thereafter, the target proteins are removed from the DNA-protein-conjugated solid supports 537 and 539 and the DNA 547 and 549 are released from the DNA-conjugated antibodies. The DNA 547 and 549 is separated from the solution and can be amplified and detected in a manner consistent with the techniques discussed in reference to FIGS. 1A and 1B.

As discussed above, each detected DNA corresponds to a particular target protein. From these results it can be determined whether one or more of the target proteins are inhibited from interacting with the first protein. Therefore, these results can then be compared to standard or known results for protein-protein interactions to determine if one or more of the target proteins were inhibited by the inhibitor protein.

Embodiment F

Another embodiment of the immuno-DNA array method/system is capable of assessing inhibitors to inhibit DNA. The immuno-DNA array system/method can be used in a kit or biosensor. In this regard, the immuno-DNA array system/method is capable of assessing if one or more inhibitors can inhibit known DNA-protein interactions. In this embodiment the immuno-DNA array system/method includes conjugating a DNA to a solid support forming DNA-conjugated solid supports. Then the DNA-conjugated solid support is contacted with a protein solution having target proteins and an inhibitor. The first DNA and the target proteins are known to interact under known incubation conditions. By adding the inhibitor, the immuno-DNA array system/method can assess whether the inhibitor inhibits the interaction of one or more of the target proteins with the first DNA.

The target proteins and inhibitor are allowed to conjugate with the first DNA forming protein-DNA-conjugated and inhibitor protein-DNA-conjugated solid supports. The solid supports are contacted with a DNA-conjugated antibody solution having multiple DNA-conjugated antibody types. The DNA bound to each DNA-conjugated antibody type is unique to that DNA-conjugate antibody type. Each type of DNA-conjugated antibody has an affinity for a particular target protein. Consequently, each DNA-conjugated antibody type conjugates with a corresponding protein on the surface of the protein-DNA-conjugated solid support, forming DNA-protein-DNA-conjugated solid supports.

Thereafter, after separating complexes from unbound molecules and DNA-conjugated antibodies, DNA associated with the DNA-conjugated antibodies is released from the DNA-conjugated antibody and separated from the solution and amplified and detected. Each type of second DNA detected corresponds to a target protein, so that detection of a type of second DNA indicates that the inhibitor proteins did not inhibit the DNA-protein interaction. The first DNA does not contain common primers, and therefore can not be amplified in the PCR step. In this manner, an assessment of whether inhibitors inhibit particular DNA-protein interactions can be conducted.

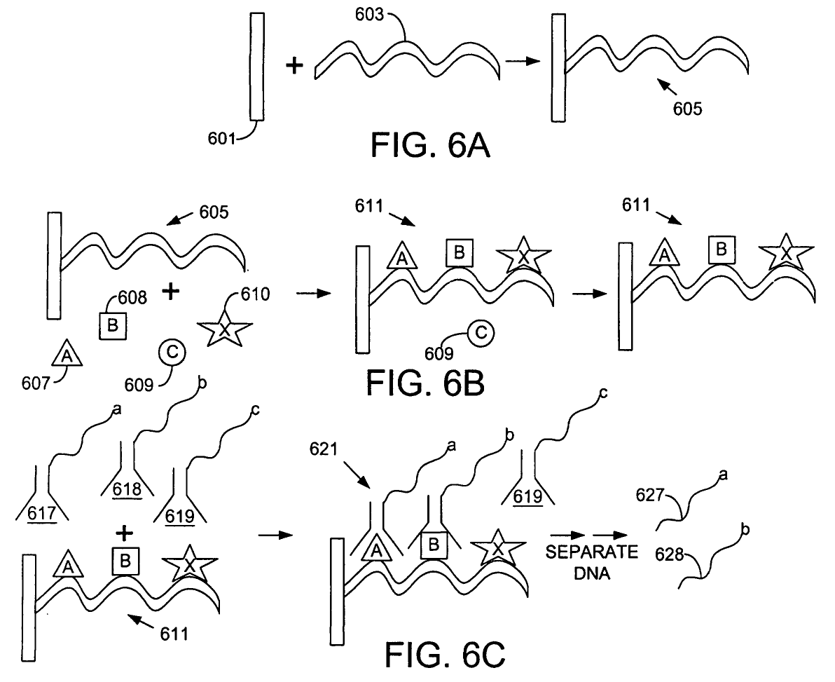

FIGS. 6A-6C are schematic diagrams that illustrate a representative embodiment of the immuno-DNA assay system/method and method that can be used to assess whether an inhibitor inhibits DNA-protein interactions. After a DNA-protein interaction is known for DNA and target proteins, an assessment can be made to determine if inhibitors inhibit the interaction between DNA and one or more target proteins.

FIG. 6A is a schematic that illustrates contacting a solid support 601 with the first DNA or DNA fragments 603 forming DNA-conjugated solid supports 605. FIG. 6B is a schematic that illustrates contacting the DNA-conjugated solid support 605 with a solution of proteins. The solution of proteins includes multiple target proteins 607, 608, and 609 and an inhibitor 610. The target proteins 607, 608, and 609 and DNA 603 exhibit conjugation under known incubation conditions. The inhibitor 610 is a protein that is being assessed to determine if it inhibits the conjugation between one or more target proteins 607, 608, and 609 and the first DNA 603. In this regard, contacting the DNA-conjugated solid supports 605 with the solution of target proteins 607, 608, and 609 and inhibitor 610 facilitates the formation of protein-DNA-conjugated solid support 611 that may include the inhibitor 610.

FIG. 6C is a schematic that illustrates contacting the protein-DNA-conjugated solid supports 611 with multiple DNA-conjugated antibody types 617, 618, and 619. Each type of the DNA-conjugated antibody 617, 618, and 619 has an affinity for one or more target proteins 607, 608, and 609, and preferably for only one target protein. Therefore, contacting the DNA-conjugated antibody types 617, 618, and 619 with the protein-DNA-conjugated solid supports 611 facilitates the bonding or binding of the DNA-conjugated antibody types 617, 618, and 619 with target protein 607, 608, and 609 to form DNA-protein-DNA-conjugated solid supports 621. The inhibitor 610 has inhibited the target protein 609 from interacting with the DNA. Consequently, DNA-conjugated antibody 619 can not conjugate to the protein-DNA conjugated solid support 611. Thereafter, the second DNA 627 and 628 are released from the DNA-conjugated antibody and separated from the solution and can be amplified and detected in a manner consistent with the techniques discussed in reference to FIGS. 1A and 1B.

As discussed above, each detected DNA corresponds to a particular target protein. From these results it can be determined whether one or more of the target proteins are inhibited from interacting with the first DNA. These results can then be compared to standard or known results for DNA-protein interactions to determine if the inhibitors inhibit one or more of the target proteins from interacting with the DNA.

Embodiment G

An embodiment of the immuno-DNA array method/system is capable of assessing the presence and levels of one or more autoantibodies. The immuno-DNA array system/method can be used in a kit or biosensor. In general, the immuno-DNA array system/method includes contacting the proteins of interest to solid supports having antibodies bound to the surface of the solid support. To this end, protein-conjugated solid supports are formed.

Then the protein-conjugated solid supports are contacted with a DNA-conjugated antibody solution having multiple DNA-conjugated antibody types to form DNA-conjugated solid supports. The DNA bound to each DNA-conjugated antibody type is unique for each DNA-conjugate antibody type. In addition, each type of DNA-conjugate antibody has an affinity for a particular protein. Consequently, each DNA-conjugated antibody type binds to a corresponding protein on the surface of the solid support.

For example, a patients serum, which may contain different types of autoantibodies is contacted with a mixture of purified proteins, cell lysate, or tissue lysate to form autoantibody-antigen complexes. The complexes are then contacted with DNA-conjugated antibodies to form autoantibody-antigen-DNA-conjugated antibody complexes. For example, anti-human IgG conjugated to solid supports are applied to separate the autoantibody-antigen-DNA-conjugated antibody complexes from excess amounts of DNA-conjugated antibody and other unbound molecules. Thereafter, the protein is removed from the solid support and the DNA is released from the DNA-conjugated antibody. The DNA is separated from the solution, amplified, and detected. The detection of a type of DNA indicates the presence of a particular protein. In this manner, an assessment of the interaction of the antigens and autoantibodies can be conducted.

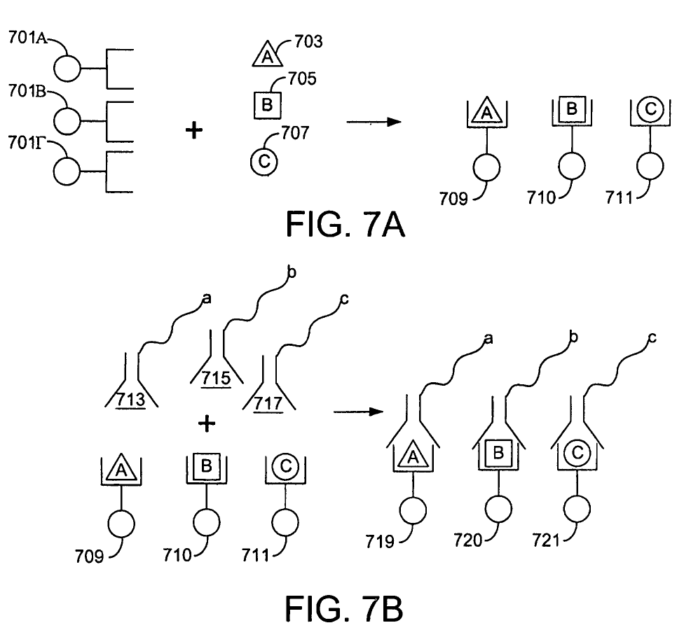

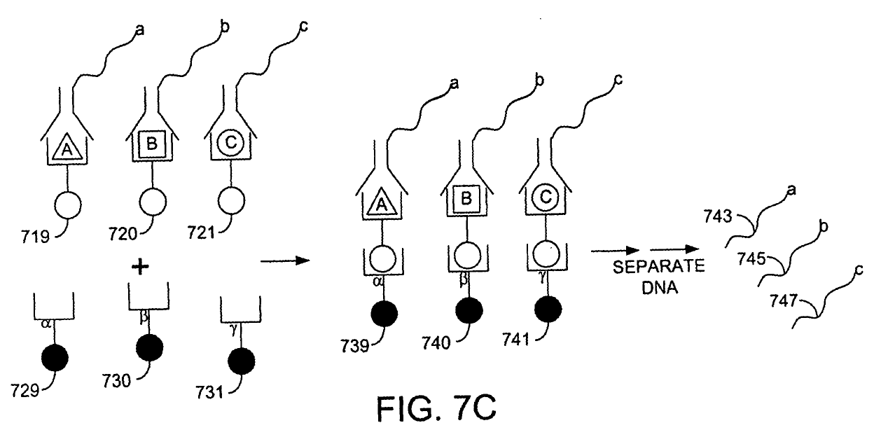

FIGS. 7A and 7B are schematic diagrams that illustrate another representative embodiment of the immuno-DNA assay system/method. FIG. 7A is a schematic that illustrates contacting multiple autoantibodies 701A, 701B, and 701Γ with a solution of proteins that includes specified proteins 703, 705, and 707. Alternatively, the autoantibodies can be attached to a substrate, as described in the examples above.

Specified proteins 703, 705, and 707 bind to corresponding autoantibodies 701A, 701B, and 701 Γ forming multiple protein (antigen)-autoantibody complexes 709, 710, and 711. The solution of autoantibody contains one and more types of autoantibodies, 701A, 701B, and 701 Γ, where each autoantibody (e.g., A, B, and Γ) has an affinity for corresponding proteins (antigens).

FIG. 7B is a schematic that illustrates contacting the protein-autoantibody complexes 709, 710, and 711 with a solution having a plurality of DNA-conjugated antibody types 713, 715 and 717. Each type of the DNA-conjugated antibody 713, 715, and 717 has an affinity for one or more specified proteins 703, 705 and 707, and preferably for only one specified protein. Therefore, contacting the DNA-conjugated antibody solution with the protein-autoantibody complexes 709, 710, and 711 facilitates the binding or bonding of each type of DNA-conjugated antibody 713, 715, and 717 with the specified proteins to form plural complexes (i.e., protein-autoantibody-DNA-conjugated antibody) containing DNA-conjugated antibody, specific protein, and autoantibody, 719, 720, and 721.

FIG. 7C is a schematic that illustrates contacting the protein-autoantibody-DNA-conjugated antibody complex 719, 720, and 721 with a solution of anti-species specific antibodies α, β, and γ (e.g., if the autoantibodies are from human serum, anti-human IgG will be used) conjugated to a solid support 729, 730, and 731. The solution of antibodies 729, 730, and 731 includes one or more types of antibodies, where each autoantibody (e.g., A, B, and Γ) has an affinity for the same corresponding antibody (α, β, and γ) (e.g., human IgG). Thus, each antibody 729, 730, and 731 binds to a corresponding autoantibody on the surface of the DNA-conjugated solid supports 719, 720, and 721. The resultant product of contacting the protein-autoantibody-DNA-conjugated antibody complex 719, 720, and 721 with the solution of antibodies 729, 730, and 731 are antibody-protein-autoantibody-DNA conjugated antibody complexes 739, 740, and 741.

Subsequently, the protein is removed from the complexes 739, 740, and 741 and the DNA 743, 745, and 747 is released from the DNA-conjugated antibodies. The DNA 743, 745, and 747 is separated from the solution and can be amplified and detected in a manner consistent with the techniques discussed in reference to FIGS. 1A and 1B.

The detection of each type of DNA indicates the presence of a particular protein.

In this manner, an assessment of the presence and levels of autoantibodies can be conducted.

Embodiment H

The following embodiment illustrates the application of the immuno-DNA microarray method/system to assess multiple protein phosphorylation. The immuno-DNA array system/method can be used in a kit or biosensor. Two well-known model systems have been selected to test the immuno-DNA microarray system/method. One model includes the use of A431 cells stimulated with EGF. Treatment of A431 cells with EGF leads to the phosphorylation of EGFR and activates the Ras signal transduction pathway.

Another model includes treatment of NIH3T3 cells with PDGF brings about the phosphorylation of PDGFR and activates Ras signal transduction pathway.

Several important proteins such as EGFR, PDGFRα and PLCγ in those signal pathways can be selected for conjugation with their corresponding cDNA obtained from EST. The EST clones are available from several vendors. When multiple EST clones are available, the EST with the shortest sequences (e.g., 200 to 1,000 bp) and high specificity can be selected since longer sequences may affect the ability of cDNA-conjugated antibodies to bind with their corresponding antigens. Since all of the EST can be amplified by the same primers, the use of EST clones can simplify the PCR process during the preparation of conjugation of antibodies to cDNA and the generation of probes for DNA microarrays. Antibodies with the highest titer and specificity can be selected for conjugation (e.g., monoclonal antibodies).

To make cDNA conjugated antibodies, the antibodies can be treated with a 10-fold molar excess of sulfo-GMBS. After removing untreated sulfo-GMBS by chromatography over a PD-10 column, the antibody can then be concentrated in a centricon. The sulfo-GMBS-activated antibody and 5'thiol cDNA can be conjugated. Antibodies conjugated to cDNA can then be purified by anion exchange chromatography on Q-Sepharose. Using a salt gradient, the free cDNA can be further removed by size exchange chromatography on Superdex-200.

Another approach to conjugate DNA to antibodies is to use aldehyde modification oligonucleotides/DNA. Proteins are modified by SHNH (Succininmidyl hydraziniumnicotinate hydrochloride) in 100 ml DMF (N,N-Dimethylformamide). Modified proteins are conjugated to aldehyde-modified oligonucleotides/DNA by incubation over night at room temperature. The reactions are assayed by DNA gel analysis. The conjugates are purified by ion-exchange DEAE Sepharose™ Fast column or Q-sephorase™ Fast Flow column. DAN-conjugated antibody fractions are collected and are subjected to DNA gel analysis and dialysis in 1×PBS (phosphate buffer salt) buffer.

The effect of the conjugation on the ability of the antibody to bind with the antigen can be determined by immunoprecipitation. Conjugated antibody and unconjugated antibody can be incubated with cell lysates prepared from growth factor (GF)-stimulated cells. The immunoprecipitated complexes can then be separated by sodium dodecyl sulphate polyacrimide gel electrophoresis (SDS PAGE).

After transferring the proteins to PDVF membranes, the membranes can be probed with unconjugated antibody and DNA-conjugated antibody, respectively. The intensities of signals can be compared. If the intensities of signals are similar between DNA-conjugated and unconjugated antibodies, this indicates that the conjugation of cDNA does not affect the ability of the antibody to bind to its antigen. If the conjugation of cDNA significantly reduces the ability of the antibody to bind its antigen, the size of cDNA can be reduced. From the immuno-PCR data, the conjugation of cDNA up to 1 to 2 kb of the antibody should not significantly affect the ability of the antibody to bind to its antigen. High sensitivity should be able to be obtained using the PCR amplification technique even if the attached DNA affects the binding activity of the antibody to the antigen.

To optimize the development of the cDNA conjugated antibodies several sets of experiments can be performed. One includes varying the concentration of the DNA-conjugated antibodies. Several concentrations such as 1 ng/ml, 10 ng/ml, 100 ng/ml, 100 μg/ml and 10 μg/ml can be used to select the optimal concentration. Another parameter that can be tested includes varying the PCR cycles. Different PCR cycles such as 10 cycles, 20 cycles, and 30 cycles can be tested. Non-PCR labels can also be used to detect the signal and are well known in the art. Since the model cell lines express high levels of either EGFR or PDGFR, different diluted cell lysates can be used to test the detection limits of this embodiment.

To make cDNA microarray membranes, EST sequences used for construction of DNA-conjugated antibodies can be amplified using a pair of universal primers. The amplified products can be checked by agarose gel electrophoresis and quantitated by OD at 260 over 280 10 picograms of DNA can be spotted onto nitrocellulose membranes. EST sequences, which do not match for the construction of DNA-conjugated antibodies can be used as a negative control.

After the DNA conjugated antibodies and mini-DNA array membranes are made, the system can be tested. The cell lysates and tumor tissue lysates can be prepared by homogenization in RIPA buffer containing appropriate proteinase and phosphatase inhibitors. Lysates can pass through a 26 gauge needle to disperse any large aggregates. Cell lysates from EGF-stimulated A431 cells and PDGF-stimulated NIH3T3 cells can be incubated with agarose-conjugated anti-phosphotyrosine antibody at about 4° C. for about 1 hour. The phosphotyrosine proteins can then be immunoprecipitated by agarose-conjugated anti-phosphotyrosine antibody.

After wash with RIPA, the immunoprecipitated complexes can be incubated with DNA conjugated specific antibodies at about 4° C. for about 1 hour to overnight. The immunoprecipitated complexes containing tyrosine-phosphorylated proteins, agarose-conjugated anti-phosphorylated tyrosine antibody and DNA-conjugated specific antibodies can be pulled down by centrifugation. The excess amounts of unbound cDNA conjugated antibodies can be washed away.

Three control experiments can be conducted to determine the effectiveness of the system and method of the present invention. One control experiment includes using agarose instead of agarose-conjugated anti-phospho-tyrosine antibody. Any signals resulting in this condition can be attributed to the unspecific binding of DNA-conjugated antibodies to agarose rather than their specific antigens or the unspecific complexes between proteins, agarose, and antibodies. Another control experiment uses H₂O instead of DNA-conjugated antibodies. The signals resulting from this experiment can be attributed to the unspecific binding of cellular DNA to the agarose-conjugated antibody. Another control experiment uses RIPA instead of cell lysate. The signals resulted from this experiment again can be attributed the unspecific binding between agarose-conjugated anti-phospho-tyrosine antibody and DNA-conjugated antibody.

The complexes can be heated at 85-100 C to set free (release) DNA from conjugated antibodies. After heating, the DNA can be recovered. The DNA can then be amplified and labeled by PCR in the presence of 33p dUTP or cy3 dUTP. The amplified products can hybridize to the DNA array membranes containing cDNA immobilized onto membrane (e.g., nitrocellulose membrane). The system can be detected by exposure to koda x-film or phospho-imaging system, chemiluminescence imaging system, or laser scanner. The recovered DNA also can hybridize the DNA chips containing cDNA or oligonucleotides. Since specific cDNAs are attached to a corresponding antibody, the intensities of signal reflect the levels of corresponding proteins. Since all of proteins are immunoprecipitated with anti-phosphotyrosine antibody, the intensities of signal reflect the phosphotyrosine status of specific proteins. Since A43 1 cells express high amount of EGFR, tyrosine phosphorylation in EGFR should be detected. NIH3T3 cells express a high amount of PDGFR, thus tyrosine phosphorylation in PDGFR can also be detected. Therefore, positive and negative signals can be detected.

One population of DNA can be labeled with cy3 (e.g., the DNA recovered from experiment using A431 cells), while another population of DNA can be labeled with cy5, (e.g., the DNA recovered from experiment using NIH3T3 cells). Both probes can then hybridize to the same cDNA array membranes or DNA chip. The signal can be scanned using a laser scanner or CCD camera and the intensities of the signals can be compared simultaneously.

The tyrosine-phosphorylated status of proteins can be confirmed and verified by immuno-Western blotting analysis. In this type of experiment, cell lysates can be immunoprecipitated with anti-phospho-tyrosine antibody. Then the immunoprecipitated complexes can be separated by SDS-PAGE. After transferring the proteins onto membranes, the membranes can be probed with specific antibodies. The results from immuno-DNA array systems/methods and immuno-Western blot can be compared, which can be used to assess the specificity and sensitivity of immuno-DNA array systems/methods.

After the cell lines are tested, the methodology can be used to assess human tumor tissues. Breast cancer tissues can be used since most of breast cancer tissues express high amounts of phosphorylated EGFR. Tumor tissue lysates can be incubated with agarose-conjugated anti-phospho-tyrosine antibody. The immunoprecipitated complexes can then be incubated with specific cDNA conjugated antibody. The recovered cDNA can then be amplified by PCR and used as probes to hybridize to DNA array membranes. The results can be confirmed by immuno-Western blot analysis.

Embodiment I

The following embodiment illustrates the application of the immuno-DNA array method/system to assess multiple protein expression levels. The immuno-DNA array system/method can be used in a kit or biosensor. Several antibodies and recombinant proteins can be selected to test the immuno-DNA microarray system/method. EGF and anti-EGF antibody, insulin and anti-insulin, MCP-1 and anti-MCP-1 can be used. The EST clones are available from several vendors. When multiple EST clones are available, those with the shortest sequences (e.g., 200 to 1,000 bp) and highest specificity can be selected since longer sequences may affect the ability of cDNA-conjugated antibodies to bind to their corresponding antigens. Since all of the ESTs can be amplified by the same primers, the use of EST clones can simplify the PCR process during the preparation of conjugated antibodies to cDNA and the generation of probes for DNA microarrays.

DNA conjugate antibodies can be generated in a manner as described above. To optimize the conditions of the array, several sets of experiments can be performed. One is the concentration of DNA-conjugated antibodies. Several concentrations, such as 1 ng/ml, 10 ng/ml, 100 ng/ml and 1000 ng/ml, will be used to select the optimal concentration. Another parameter that can be tested includes using different PCR cycles. Different cycles such as 10 cycles, 20 cycles and 30 cycles can be tested.

To make mini-cDNA microarray chips, EST sequences used for construction of DNA-conjugated antibodies can be amplified using a pair of universal primers. The amplified products can be checked by agarose gel electrophoresis and quantitated by OD at 260 over 280 50 pg of DNA can be spotted onto Parckard Hydrogel chip or any other types of glass slides. EST sequences that do not match for the construction of DNA-conjugated antibodies can be used as negative control.

After the DNA conjugated antibodies and mini-DNA array chips are made, the system can be tested. Purified recombinant proteins (100 nanograms of protein) can be conjugated to magnetic beads. Bead-conjugated proteins can then be incubated with DNA-conjugated antibodies at about 4° C. for about 2 hours. Excess DNA-conjugated antibodies can be removed by magnetic field and washed with PBS. A control experiment can use magnetic beads rather than magnetic bead conjugated proteins. The signals resulting from this experiment can contribute to the unspecific binding between magnetic beads and DNA-conjugated antibodies. The complexes can be digested with proteinase K to remove the proteins and release DNA from conjugated antibodies. After passing through ultrafree-probind column (Millipore) to remove proteins and small peptides, the DNA can be recovered by ethanol precipitation. The DNA can then be amplified and labeled by PCR in the presence of cy3 dUTP. The amplified products can hybridize to the mini-DNA array chips containing corresponding cDNA. Then the signal can be scanned using a laser scanner.

Other immobilization approaches such as conjugation of proteins to agarose or to PDVF membranes can be tested. From this set of experiments, the immobilization condition that produces the highest signals and that is easiest to perform can be selected.

The detection sensitivity of this approach can be analyzed. Different amounts of purified recombinant protein can be used to test the detection sensitivity. In addition, inter-chip variability and intra-chip variability can be determined.

The next stage of development can focus on assessing multiple antibodies. Approximately 1000 antibodies, which play important roles in signal transduction, cell growth control, DNA repair and apoptosis, can be selected. Specific cDNA-conjugated antibodies can be generated. Antibodies with high titer and specificity (monoclonal antibodies) can be selected for conjugation to DNA. The system can be analyzed using cell lysates and tissue lysates. The immuno-DNA array system/method should be able to simultaneously detect at least 1000 proteins with high specificity and sensitivity.

The differential expression of proteins between normal mammary gland and breast cancer tissue can be examined by immuno-DNA array system/method. One hundred micrograms of total tissue lysates prepared from normal mammary gland and breast cancer tissue can be conjugated to magnetic beads, respectively. Bead-conjugated tissue lysates can then be immunoprecipitated with a mixture of DNA-conjugated antibodies. Unbound antibodies can then be removed by magnetic field and proteins can be removed by proteinase digestion. DNA can be recovered by precipitation. One pool of DNA (e.g., normal mammary gland) can be labeled with cy3 and another pool of DNA (e.g., breast cancer tissue) can be labeled with cy5. The two pools can be combined and hybridized with DNA chips. Signals can be scanned and analyzed and the expression pattern can be classified by clustering. Several differential proteins can be further confirmed by Western blot analysis.

It should be emphasized that the above-described embodiments of the present invention, particularly, any “preferred” embodiments, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

1. A method of detecting proteins, comprising:
exposing a solid support to a solution containing proteins;
conjugating proteins to the solid support;
exposing the solid support to a plurality of types of DNA-conjugated antibodies, wherein each type of DNA-conjugated antibodies has an affinity for a specified protein;
forming a complex between a protein conjugated with the solid support and a type of DNA-conjugated antibody when the protein is the specified protein for which the DNA-conjugated antibody has an affinity;
separating the complex from the solution of proteins and the DNA-conjugated antibodies;
releasing DNA from the DNA-conjugated antibodies; and
detecting the DNA, wherein each DNA indicates the presence of the specified protein.

2. The method of claim 1, wherein the proteins are modified-proteins.

3. The method of claim 1, wherein detecting the DNA further comprises:
amplifying the DNA using techniques selected from polymerase chain reaction (PCR) techniques, rolling circle amplification (RCA), techniques using biotinyl tyramide and hydrogen peroxide, and 3 DNA-label techniques.

4. The method of claim 1, wherein detecting the DNA further comprises:
detecting the DNA using techniques selected from fluorescence, chemiluminescence, substrate staining, isotope detection, surface plasmon resonance, resonance light scattering, electronic sensor, magnetic sensor, and microcantilevering.

5. The method of claim 1, wherein the solid support is selected from magnetic beads, agarose, membranes, sepharose, glass slides, and tissue culture plates.

6. The method of claim 1, wherein releasing DNA comprises using techniques selected from protease digestion, proteinase digestion, phenol extraction, chloroform extraction, and heat.

7. The method of claim 1, wherein the antibodies in the plurality of DNA-conjugated antibodies can be substituted with compounds selected from DNA, RNA, lectins, hormones, carbohydrates, lipids, small molecules, cells, drugs, and other capture reagents.

8. A method of detecting protein-protein interactions, comprising:
exposing a solid support to a first solution having a plurality of first proteins, wherein the plurality of first proteins conjugate with the solid support;
exposing the solid support to a second solution of proteins, wherein the proteins in the second solution maybe capable of conjugating with the plurality of first proteins;
exposing the solid support to a plurality of types of DNA-conjugated antibodies, wherein each type of DNA-conjugated antibody has an affinity for a specified protein;
forming a complex between a protein of the second solution that has conjugated with the first protein and a type of DNA-conjugated antibody when the protein of the second solution is the specified protein for which the DNA-conjugated antibody has an affinity;
separating the complex from the solution of first proteins, second proteins, and the DNA-conjugated antibodies;
releasing DNA from the DNA-conjugated antibodies; and
detecting the DNA, wherein each DNA detected indicates the presence of the specified protein, which indicates that the specified proteins interacted with the first protein.

9. The method of claim 8, wherein the solution of proteins contains inhibitor compounds that inhibit the interaction of specified proteins with the first protein.

10. The method of claim 8, wherein the proteins are modified proteins.

11. The method of claim 8, wherein detecting the DNA further comprises:
amplifying the DNA using techniques selected from polymerase chain reaction (PCR) techniques, rolling circle amplification (RCA), techniques using biotinyl tyramide and hydrogen peroxide, and 3 DNA-label techniques.

12. The method of claim 8, wherein detecting the DNA further comprises:
detecting the DNA using techniques selected from fluorescence, chemiluminescence, substrate staining, isotope detection, surface plasmon resonance, resonance light scattering, electronic sensor, magnetic sensor, and microcantilevering.

13. The method of claim 8, wherein the solid support is selected from magnetic beads, agarose, membranes, sepharose, glass slides, and tissue culture plates.

14. The method of claim 8, wherein releasing DNA comprises using techniques selected from protease digestion, proteinase digestion, phenol extraction, chloroform extraction, and heat.

15. The method of claim 8, wherein the antibodies in the plurality of DNA-conjugated antibodies can be substituted with compounds selected from DNA, RNA, lectins, hormones, carbohydrates, lipids, small molecules, cells, drugs, and other capture reagents.

16. A method of detecting protein-DNA interactions, comprising:
exposing a solid support to a first DNA having at least one portion, wherein the first DNA conjugates with the solid support;
exposing the solid support to a solution of proteins, wherein the proteins in the solution are capable of conjugating with a portion of the first DNA;
exposing the solid support to a plurality of types of DNA-conjugated antibodies, wherein each type of DNA-conjugated antibody has an affinity for a specified protein;
forming a complex between a protein in the solution that has conjugated with a portion of the first DNA and a type of DNA-conjugated antibody when the protein is the specified protein for which the DNA-conjugated antibody has an affinity;
separating the complex from the solution of proteins and the DNA-conjugated antibodies;
releasing DNA from the DNA-conjugated antibodies; and
detecting the DNA, wherein each DNA detected indicates the presence of the specified proteins, which indicates that the specified proteins interacted with the portion of DNA.

17. The method of claim 16, wherein the solution of proteins contains inhibitor compounds that are capable of inhibiting the interaction of a specified protein with the first DNA.

18. The method of claim 16, wherein the proteins are modified proteins.

19. The method of claim 16, wherein detecting the DNA further comprises:
amplifying the DNA using techniques selected from polymerase chain reaction (PCR) techniques, rolling circle amplification (RCA), techniques using biotinyl tyramide and hydrogen peroxide, and 3 DNA-label techniques.

20. The method of claim 16, wherein detecting the DNA further comprises:
detecting the DNA using techniques selected from fluorescence, chemiluminescence, substrate staining, isotope detection, surface plasmon resonance, resonance light scattering, electronic sensor, magnetic sensor, and microcantilevering.

21. The method of claim 16, wherein the solid support is selected from magnetic beads, agarose, membranes, sepharose, glass slides, and tissue culture plates.

22. The method of claim 16, wherein releasing DNA comprises using techniques selected from protease digestion, proteinase digestion, phenol extraction, chloroform extraction, and heat.

23. The method of claim 16, wherein the antibodies in the plurality of DNA-conjugated antibodies can be substituted with compounds selected from DNA, RNA, lectins, hormones, carbohydrates, lipids, small molecules, cells, drugs, and other capture reagents.

24. The method of claim 16, wherein the first DNA can be a polypeptide.

25. A method of detecting modified-proteins, comprising:
exposing a solid support to a solution containing modified-proteins;
conjugating modified-proteins to the solid support;
exposing the solid support to a plurality of types of DNA-conjugated antibodies, wherein each type of DNA-conjugated antibody has an affinity for a specified modified-protein;
forming a complex between a modified-protein conjugated to the solid support and a type of DNA-conjugated antibody when the modified-protein is the specified modified-protein for which the DNA-conjugated antibody has an affinity;
separating the complex from the solution of modified-proteins and the DNA-conjugated antibodies;
releasing DNA from the DNA-conjugated antibodies; and
detecting the DNA, wherein each DNA detected indicates the presence of the specified modified-proteins.

26. The method of claim 25, wherein the modified-protein is modified by a modification process selected from phosphorylation, glycosylation, oxidation, ubiquitination, and acetylation.

27. The method of claim 25, wherein detecting the DNA further comprises:
amplifying the DNA using techniques selected from polymerase chain reaction (PCR) techniques, rolling circle amplification (RCA), techniques using biotinyl tyramide and hydrogen peroxide, and 3 DNA-label techniques.

28. The method of claim 25, wherein detecting the DNA further comprises:
detecting the DNA using techniques selected from fluorescence, chemiluminescence, substrate staining, isotope detection, surface plasmon resonance, resonance light scattering, electronic sensor, magnetic sensor, and microcantilevering.

29. The method of claim 25, wherein the solid support is selected from magnetic beads, agarose, membranes, sepharose, glass slides, and tissue culture plates.

30. The method of claim 25, wherein removing DNA comprises using techniques selected from protease digestion, proteinase digestion, phenol extraction, chloroform extraction, and heat.

31. The method of claim 25, wherein the antibodies in the plurality of DNA-conjugated antibodies can be substituted with compounds selected from DNA, RNA, lectins, hormones, carbohydrates, lipids, small molecules, cells, drugs, and other capture reagents.

32. A method of detecting autoantibodies, comprising:
exposing a solution containing proteins to a solution containing a plurality of autoantibodies, wherein each autoantibody has an affinity for a specified protein;
forming a first complex between a protein in the solution and a type of autoantibody when the protein is the specified protein for which the autoantibody has an affinity;
exposing the first complex to a solution containing a plurality of types of DNA-conjugated antibodies, wherein each type of DNA-conjugated antibody has an affinity for a specified protein;
forming a second complex between first complex and a type of DNA-conjugated antibody when the protein is the specified protein for which the DNA-conjugated antibody has an affinity;
exposing the second complex to a solution having a plurality of antibodies, wherein each type of antibody has an affinity for a specified autoantibody;
forming a third complex between the second complex and a type of antibody when the autoantibody is the specified antibody for which the antibody has an affinity;
separating the third complex from the solution;
releasing DNA from the DNA-conjugated antibodies; and
detecting the DNA, wherein each DNA detected indicates the presence of a specified autoantibody.

33. The method of claim 32, wherein detecting the DNA further comprises:
amplifying the DNA using techniques selected from polymerase chain reaction (PCR) techniques, rolling circle amplification (RCA), techniques using biotinyl tyramide and hydrogen peroxide, and 3 DNA-label techniques.

34. The method of claim 32, wherein detecting the DNA further comprises:
detecting the DNA using techniques selected from fluorescence, chemiluminescence, substrate staining, isotope detection, surface plasmon resonance, resonance light scattering, electronic sensor, magnetic sensor, and microcantilevering.

35. The method of claim 32, wherein releasing DNA further is executed using techniques selected from protease digestion, proteinase digestion, phenol extraction, chloroform extraction, and heat.

36. A method of detecting protein-small molecule interactions, comprising:
exposing a support to a plurality small molecules, wherein the plurality of small molecules conjugate with the support;
exposing the support to a solution of proteins, wherein each of the proteins in the second solution are capable of conjugating with a specified small molecule;
forming a first complex between the a small molecule and a type of protein when the small molecule is the specified small molecule for which the protein has an affinity;
exposing the support to a plurality of types of DNA-conjugated antibodies, wherein each type of DNA-conjugated antibodies has an affinity for a specified protein;
forming a second complex between a protein conjugated to the support and a type of DNA-conjugated antibody when the protein is the specified protein for which the antibody has an affinity;
separating the second complex from the solution of proteins and the DNA-14 conjugated antibodies;
removing DNA from the DNA-conjugated antibodies; and
detecting the DNA, wherein each DNA detected indicates the presence of the specified proteins, which indicates that the specified proteins interacted with the small molecules.

37. The method of claim 36, wherein detecting the DNA further comprises:
amplifying the DNA using techniques selected from polymerase chain reaction (PCR) techniques, rolling circle amplification (RCA), techniques using biotinyl tyramide and hydrogen peroxide, and 3 DNA-label techniques.

38. The method of claim 36, wherein detecting the DNA further comprises:
detecting the DNA using techniques selected from fluorescence, chemiluminescence, substrate staining, isotope detection, surface plasmon resonance, resonance light scattering, electronic sensor, magnetic sensor, and microcantilevering.

39. The method of claim 36, wherein releasing DNA comprises using techniques selected from protease digestion, proteinase digestion, phenol extraction, chloroform extraction, and heat.

40. A kit for use in a method according to claim 1, the kit comprising the reageants and instructions for the performance of the method and interpretation of the results.

41. A kit for use in a method according to claim 16, the kit comprising the reageants and instructions for the performance of the method and interpretation of the results.

42. A kit for use in a method according to claim 25, the kit comprising the reageants and instructions for the performance of the method and interpretation of the results.

43. A kit for use in a method according to claim 32, the kit comprising the reageants and instructions for the performance of the method and interpretation of the results.

44. A kit for use in a method according to claim 36, the kit comprising the reageants and instructions for the performance of the method and interpretation of the results.

46. An assay for detecting proteins, comprising the method claim 1.

47. An assay for detecting protein-protein interactions, comprising the method claim 8.

48. An assay for detecting protein-DNA interactions, comprising the method claim 16.

49. An assay for detecting modified-proteins, comprising the method claim 25.

50. An assay for detecting autoantibodies, comprising the method claim 32.

51. An assay for detecting protein-small molecule interactions, comprising the method claim 36.
